(19) United States
(12) Patent Application Publication
(10) Pub. No.: US 2024/0058150 A1
(43) Pub. Date: Feb. 22, 2024

(54) Arm Stabilization Devices and Methods

(71) Applicant: Proven Design Lab LLC, SAN DIEGO, CA (US)

(72) Inventors: Arni Thor, Coronado, CA (US); Matthew Provencher, Edwards, CO (US); Tyler Joseph Zajac, Vail, CO (US)

(21) Appl. No.: 18/491,747

(22) Filed: Oct. 20, 2023

Related U.S. Application Data
(63) Continuation-in-part of application No. 17/721,341, filed on Apr. 14, 2022.
(60) Provisional application No. 63/174,685, filed on Apr. 14, 2021.

(51) Int. Cl. A61F 5/01

(52) U.S. Cl. CPC A61F 5/0118

(57) ABSTRACT

An arm stabilization system designed to provide enhanced support for the human arm. The system consists of a lateral support element that contacts the side of a torso, a first arm support element that contacts the upper arm, and a second arm support element that contacts the lower arm. A plurality of subframes embedded within the support elements provide support, stability, and a consistent shape to the arm stabilization system. A novel series of connections among these subframes provides enhanced adjustability and movement options for the wearer.

### CROSS-REFERENCE TO RELATED APPLICATIONS

This patent application is a Continuation-In-Part of U.S. Non-Provisional patent application Ser. No. 17/721,341, titled “SHOULDER SUPPORT DEVICES AND METHODS,” filed on Apr. 14, 2022, which claims the benefit and priority of U.S. Provisional Patent Application No. 63/174,685, titled “SHOULDER SUPPORT DEVICES AND METHODS OF USING THE SAME,” filed Apr. 14, 2021, the contents of which are incorporated by reference in their entirety.

### BACKGROUND

### Field of the Invention

The present invention pertains generally to the field of medicine, and more particularly to an orthopedic device which holds the arm of a person in a desired position to promote healing after an injury or surgery.

### Scope of the Prior Art

Orthopedic devices such as braces and slings are well known in the art. These devices are temporarily used to support and immobilize a part of the body after an injury or surgery. For example, in the case of shoulder surgery, it is advantageous to restrict arm movement during the healing process. This often includes keeping the arm in an outward and upward “abducted” position away from the body.

The related art includes, for instance, braces for the immobilization of shoulders for wearers who have had shoulder surgery, who have injured a shoulder, or who otherwise require restriction of shoulder movement. For instance, prior art shoulder braces include embodiments known as a “gunslinger” brace and an “airplane” brace. However, prior art braces commonly restrict positioning of one type or direction. Moreover, prior art braces are not capable of large adjustment of shoulder rotation positioning as well as large adjustment of shoulder abduction-adduction positioning. For example, the Abduction Rotation Control (“ARC”) Shoulder Brace from Bledsoe Brace Systems allows minimal adjustment of abduction, ranging from adduction to about 5 to 10 degrees of abduction, and minimal adjustment of rotation, ranging between up to 15 degrees of internal rotation and up to 15 degrees of external rotation. Patients needing external rotation beyond 15 degrees are directed to use Bledsoe's ARC-XR brace. Although the ARC-XR brace allows for increased external rotation, it still does not allow increased abduction adjustment. Conversely, prior art Quadrant® “airplane” braces have abduction hinges to fix abduction positioning, but without fixing, or even sometimes restricting, rotation. Likewise, Acro Assist Arm Abduction Orthosis 50A1 enables functional shoulder positioning with arm abduction at 30, 60, or 90 degrees, but does not provide shoulder rotation positioning adjustment. As such, the prior art uses multiple braces to achieve multiple positions, often using a particular brace to achieve a particular position.

Another common issue with most if not all rigid shoulder braces are that they intend to immobilize the entire arm, keeping the elbow at 90 and the wrist fixed. This can cause pressure on the ulnar nerve and swelling builds up in the arm and hand, causing pain and discomfort. In addition, a patient with his entire arm immobilized struggles with ADLs. Having a device that can control multiple directions at the same time while also having the ability to free up the elbow and wrist is advantageous.

### SUMMARY

One aspect of the present invention is directed at an adjustable arm stabilization system designed to provide enhanced support for the human arm. The system consists of a lateral support element that contacts the side of a person's torso, a first arm support element that contacts the upper arm, and a second arm support element that contacts the lower arm. A plurality of subframes embedded within these support elements provide support, stability, and a consistent shape to the arm stabilization system.

The third subframe, rotatably attached to the second subframe, can rotate around two axes. The first axis is defined by a line passing through the multiaxial joint and the distal end of the fourth subframe. The second axis, perpendicular to the first, is defined by a horizontal line passing through the joint. Rotation around the first axis allows for internal and external rotation of an arm while rotation around the second axis allows for arm abduction and adduction. There may be locking mechanisms to restrict rotation around the two axes respectively.

The system employs shape-maintaining moldable straps. The lateral support element attaches to the waist using one such strap, and the upper and lower arm support elements attach to the upper and lower arms using two other such straps.

The system is reconfigurable and can be adjusted to support the opposite side of the torso and upper arm.

Another aspect of the present invention is directed at a method of stabilizing the arm. The provided method starts with setting up the system as described. At an intermediate time, the user can detach the fourth support element from the third in order to remove the second arm support element. Additionally, a sixth support element can be slidably attached to the third support element for further adjustability.

### BRIEF DESCRIPTION OF THE DRAWINGS

The foregoing summary, as well as the following detailed description of preferred variations of the invention, will be better understood when read in conjunction with the appended drawings. For the purpose of illustrating the invention, there is shown in the drawings variations that are presently preferred. It should be understood, however, that the invention is not limited to the precise arrangements shown. In the drawings, where:

FIGS. 1a-1b show an arm stabilization system, according to a first and second embodiment.

FIGS. 2a-2b show a lateral support element in a first and second configuration.

FIGS. 3a-3b show a rigid frame, according to a first and second embodiment.

FIGS. 4a-4e show various configurations of the rigid frame.

FIG. 5 shows a coaxial joint in between second and third subframes, according to an embodiment.

FIG. 6 shows a method of stabilizing an arm, according to an embodiment.

FIG. 7 shows an arm stabilization system, according to a third embodiment.

FIGS. 8a-8d show an adjustable abduction wedge, according to an embodiment.

### DETAILED DESCRIPTION

Implementations of the present technology will now be described in detail with reference to the drawings, which are provided as illustrative examples so as to enable those skilled in the art to practice the technology. Notably, the figures and examples below are not meant to limit the scope of the present disclosure to any single implementation or implementations, but other implementations are possible by way of interchange of, substitution of, or combination with some or all of the described or illustrated elements. Wherever convenient, the same reference numbers will be used throughout the drawings to refer to same or like parts.

Moreover, while embodiments described herein are primarily discussed in the context of arm stabilization systems and methods, it will be recognized by those of ordinary skill that the present disclosure is not so limited. In fact, the principles of the present disclosure described herein may be readily applied to the stabilization of other parts of the human anatomy. For example, many common knee or elbow injuries require the stabilization of a joint or appendage. Accordingly, the principles described herein may be readily adapted for use on the arm, elbow, ankle, etc., where movement, whether in extension, flexion, adduction, abduction, internal rotation, or external rotation, may need to be constrained in order to facilitate recovery from, for example, an injury or other medical condition, as well as to reduce pain or to help provide bio feedback to train muscles in order to prevent future injuries.

In the present specification, an implementation showing a singular component should not be considered limiting; rather, the disclosure is intended to encompass other implementations including a plurality of the same component, and vice-versa, unless explicitly stated otherwise herein. Further, the present disclosure encompasses present and future known equivalents to the components referred to herein by way of illustration.

It will be recognized that while certain aspects of the technology are described in terms of a specific sequence of steps of a method, these descriptions are only illustrative of the broader methods of the disclosure and may be modified as required by the particular application. Certain steps may be rendered unnecessary or optional under certain circumstances. Additionally, certain steps or functionality may be added to the disclosed implementations, or the order of performance of two or more steps permuted. All such variations are considered to be encompassed within the disclosure disclosed and claimed herein.

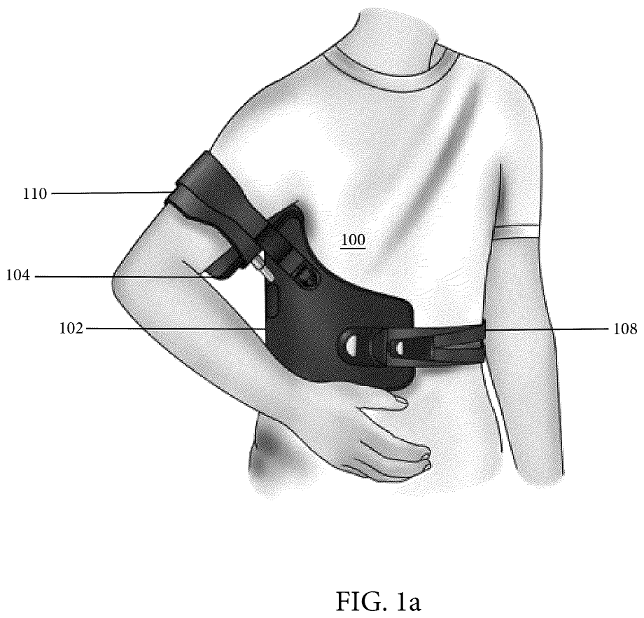

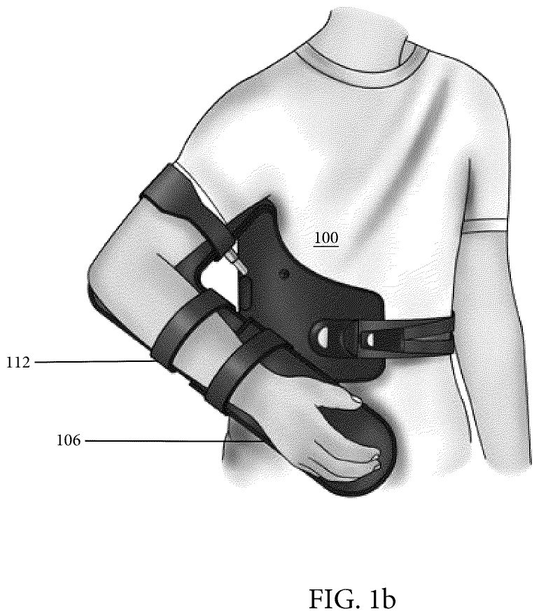

FIGS. 1a-1b show an arm stabilization system 100, according to a first and second embodiment. The arm stabilization 100 may be comprised of a lateral support element 102, a first arm support element 104, and a second arm support element 106.

The lateral support element 102 may be a bolster, lateral paddle, plate, or any other malleable or semi-rigid element that is configured to contact the side of a torso, preferably at the thorax or waist, providing proper arm positioning, and taking the weight of the arm. In an embodiment, the lateral support element 102 is securable around the waist. Preferably, the first end of a waist strap 108 is attached, permanently or removably, to a posterior end of the of the lateral support element 102 and the second end of the waist strap 108 is attachable to the anterior end of the lateral support element 102.

The first arm support element 104 may be a pad, plate, or any other malleable or semi-rigid element that is configured to contact an upper arm, providing support to the upper arm. In an embodiment, the first arm support element 104 is securable around the upper arm. Preferably, the first end of an upper arm strap 110 is attached, permanently or removably, to a first end of the first arm support element 104 and the second end of the upper arm strap 110 is attachable to a second end of the first arm support element 104.

The second arm support element 106 may be a pad, plate, or any other malleable or semi-rigid element that is configured to contact a lower arm, providing support to the lower arm. In an embodiment, the second arm support element 106 is securable around the upper arm. Preferably, the first end of a lower arm strap 112 is attached, permanently or removably, to a first end of the second arm support element 106 and the second end of the lower arm strap 112 is attachable to a second end of the second arm support element 106.

In an embodiment (not shown), the upper arm strap 110 is replaced with a wide cuff that encircles the upper arm. The cuff is configured to receive first ends of a first and second strap. The path of the first strap starts on the cuff (preferably on an anterior of the cuff), spirals clockwise around the cuff (preferably at least one full rotation around the cuff), and terminates on the lateral support element 102 (preferably on an upper anterior of the lateral support element 102). The path of the second strap starts on the cuff (preferably on an anterior of the cuff), spirals counterclockwise around the cuff (preferably at least one full rotation around the cuff), travels across the back, around the side of the torso opposite to the lateral support element 102, across the front, and terminates on the lateral support element (preferably on a lower anterior of the lateral support element 102).

For strapping arrangements described in this document and any priority documents, it should be appreciated that the path of any strap can continue to encircle the wearer for any number of rounds, or half-rounds, around his or her torso. For example, the path of a strap may continue across the front of a torso, around a side of a torso, across a back of a torso, around an opposite side of a torso, and so on. Said coiling may provide the strap with additional advantageous tensive properties.

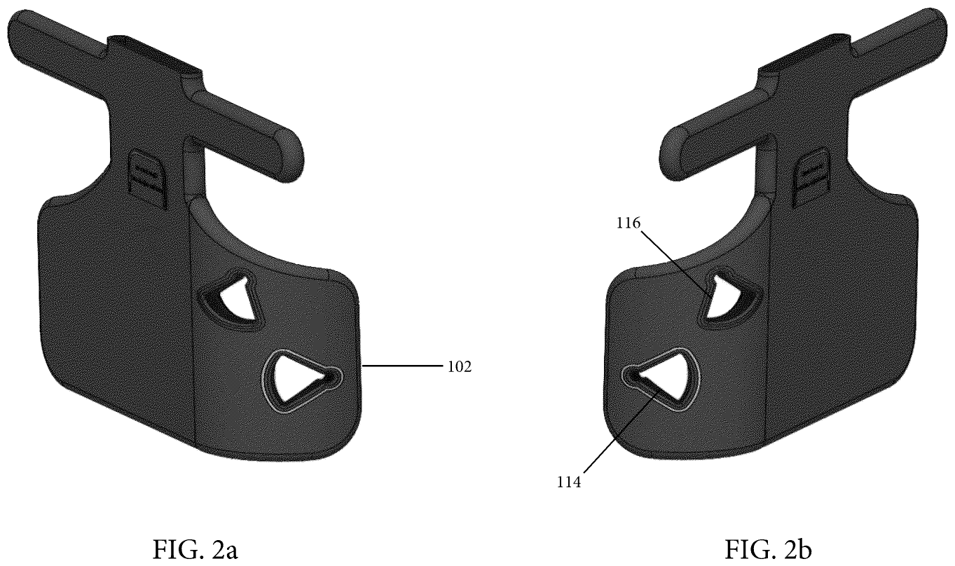

FIGS. 2a-2b show the lateral support element 102 in a first and second configuration, where the second configuration resembles the first configuration reflected across the sagittal plane. Being malleable or semi-rigid, the lateral support element 102 may be configured to contact the left or right side of a torso depending on user needs. Likewise, the first arm support element 104 may be configured to contact the left or right upper arm (mirrored configurations not shown) and the second arm support element 106 may be configured to contact the left or right lower arm (mirrored configurations not shown). As evidenced, the arm stabilization system 100 may be used to stabilize the left or right arm depending on part configuration (mirrored configurations not shown).

The lateral support element 102 may comprise a first attachment area 114 and, optionally, a second attachment area 116. The first attachment area 114, located on the anterior of the lateral support element 102, is configured to receive the second end of the waist strap 108. The second attachment area 116, located on the anterior of the lateral support element 102, is configured to receive the second end of a shoulder strap (not shown). The path of the shoulder strap may start on the posterior of the lateral support element 102, travel over the torso, and terminate at the second attachment area 116.

Preferably, the waist strap 108, upper arm strap 110, and lower arm strap 112, are moldable and shape-maintaining. Such functionality can be achieved by embedding an aluminum sheet within layers of foam padding. Moldable and shape-maintaining straps provide personalized comfort and one-handed operation of the arm stabilization system 100 as there is reduced strap movement when the system 100 is put on, taken off, or adjusted.

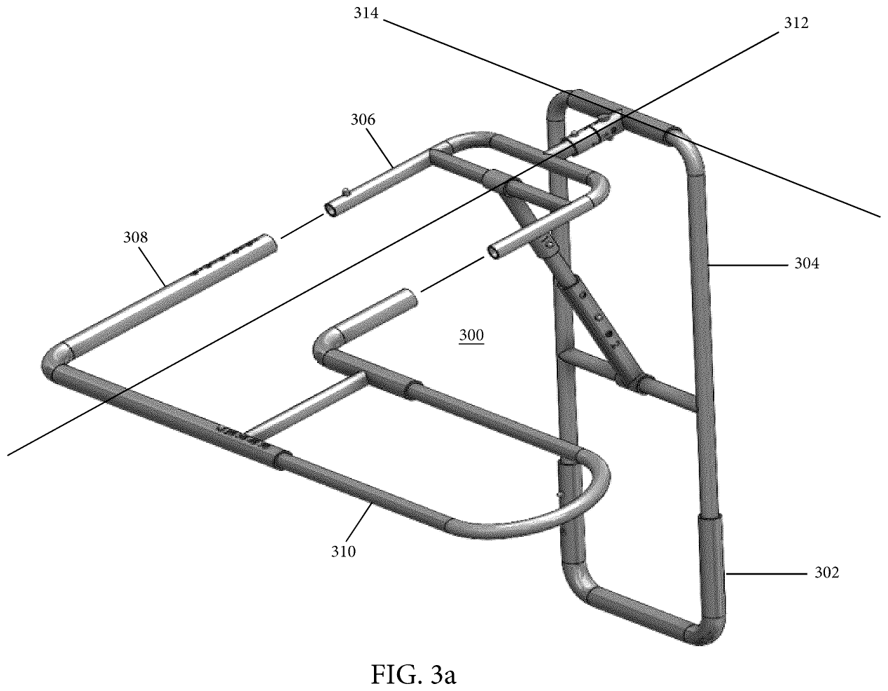

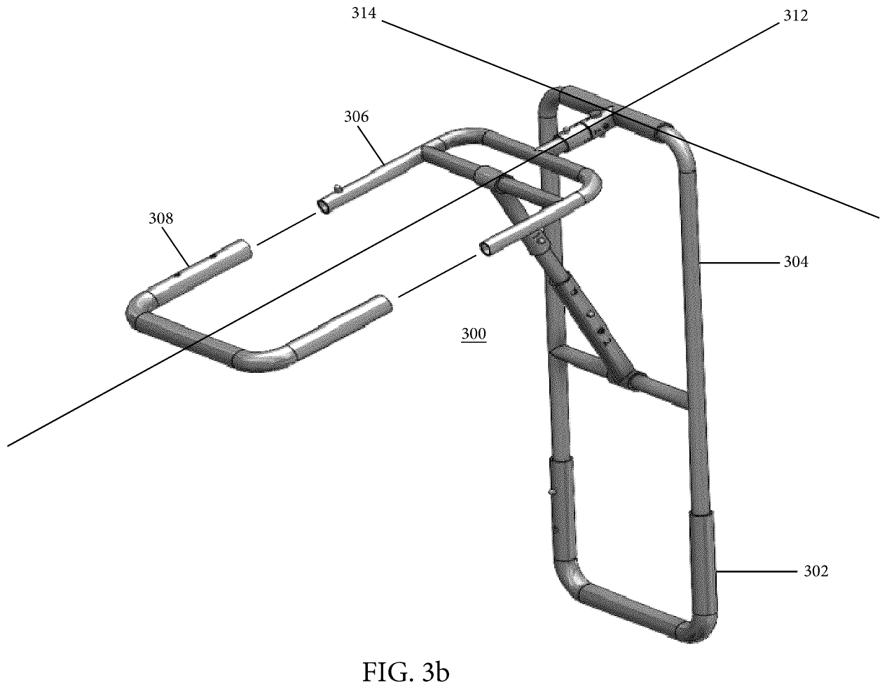

FIGS. 3a-3b show a rigid frame 300, according to a first and second embodiment. The rigid frame 300 serves as a foundational structure that provides support, stability, and a consistent shape to the arm stabilization system 100.

The rigid frame 300 may be comprised of a first subframe 302, a second subframe 304, a third subframe 306, a fourth subframe 308, and, optionally, a fifth subframe 310.

The first subframe 302 may be, permanently or removably attached to, embedded, or otherwise integrated into a lower portion of the lateral support element 102.

The second subframe 304 may be, permanently or removably attached to, embedded, or otherwise integrated into an upper portion of the lateral support element 102. The second subframe 304 is slidably attached to the first subframe 302 such that the distance between the first and second subframes is adjustable. Said functionality allows the height of the lateral support element 102 to be adjusted for a comfortable fit.

The third subframe 306 may be, permanently or removably attached to, embedded, or otherwise integrated into a proximal portion of the first arm support element 104. The third subframe 306 is rotatably attached to the second subframe 304 such that the third subframe 306 can rotate around a first axis 312 and a second axis 314. The first axis 312 is defined by a line passing through a multiaxial joint 316 and a distal end of the fourth subframe 308. Rotation around the first axis 312 allows for internal and external rotation of an arm. The second axis 314, perpendicular to the first axis 312, is defined by a horizontal line passing through the multiaxial joint 316. Rotation around the second axis 314 allows for arm abduction and adduction.

The fourth subframe 308 may be, permanently or removably attached to, embedded, or otherwise integrated into a distal portion of the first arm support element 104. The fourth subframe 308 is slidably attached to the third subframe 306 such that the distance between the third and fourth subframes is adjustable. Said functionality allows the length of the first arm support element 104 to be adjusted for a comfortable fit.

The fifth subframe 310 may be, permanently or removably attached to, embedded, or otherwise integrated into the second arm support element 106. The fifth subframe 310 is slidably attached to the fourth subframe 306 such that the distance between the fourth and fifth subframes is adjustable. Said functionality allows the length of the second arm support element 106 to be adjusted for a comfortable fit.

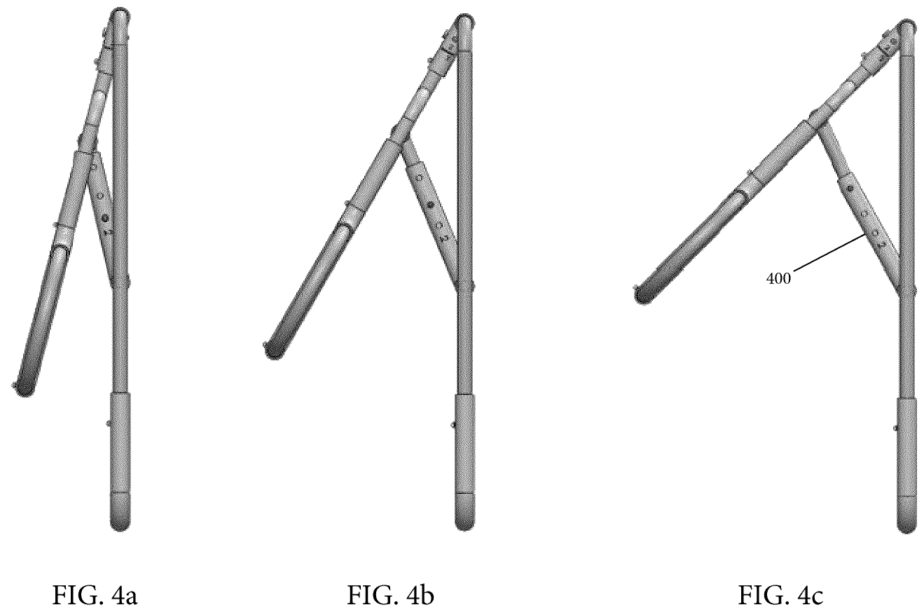

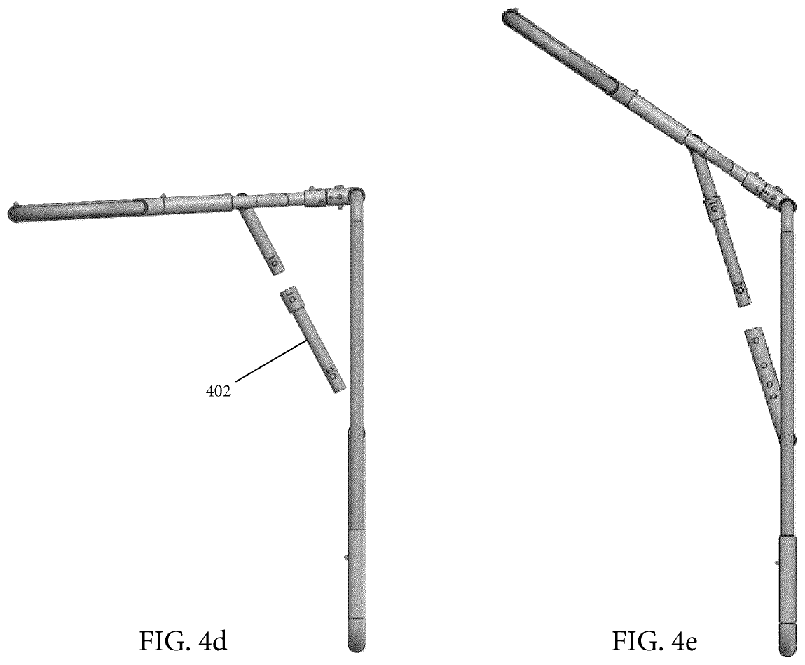

FIGS. 4a-4e show various configurations of the rigid frame 300. Namely, FIG. 4a shows a 15-degree angle in between the second 304 and third 306 subframes, FIG. 4b shows a 30-degree angle in between the second 304 and third 306 subframes, and FIG. 4c shows a 45-degree angle in between the second 304 and third 306 subframes. The desired angle is chosen by adjusting a length of the connecting element 400, where the first end of the connecting element 400 is permanently or removably attached to the second subframe 304 and the second end of the connecting element 400 is permanently or removably attached to the third subframe 306.

To achieve larger angles in between the second 304 and third 306 subframes, such as the those shown in FIG. 4d and FIG. 4e, the length of the connecting element 400 can be extended via the addition of an extension piece 402 or a plurality of extension pieces 402.

It should be appreciated that the angle in between the second 304 and third 306 subframes can be adjusted from 0-360 degrees. Said functionality allows the same rigid frame 300 to be used for both right and left arm stabilization, depending on the configuration.

The connecting element 400 serves as a locking mechanism such that, when attached to the second 304 and third 306 subframes, the connecting element 400 prevents rotation of the third subframe 306 around the second axis, hindering arm abduction and adduction.

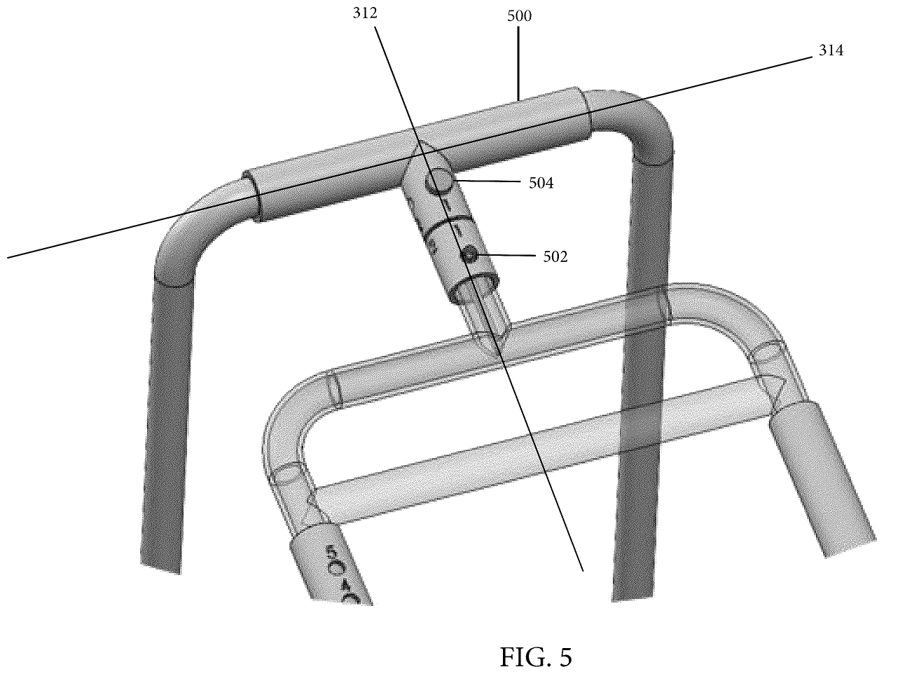

FIG. 5 shows a coaxial joint 500 in between the second 304 and third 306 subframes. The coaxial joint 500 allows the third subframe 306 to rotate around the first axis 312 and the second axis 314. Rotation around the first axis 312 allows for internal and external rotation of an arm while rotation around the second axis 314 allows for arm abduction and adduction.

The button spring 502, in conjunction with the quick release pin 504, serves as a locking mechanism such that, when the button spring 502 is not engaged, the quick release pin 502 prevents the rotation of the third subframe around the first axis, hindering the internal and external rotation of the arm.

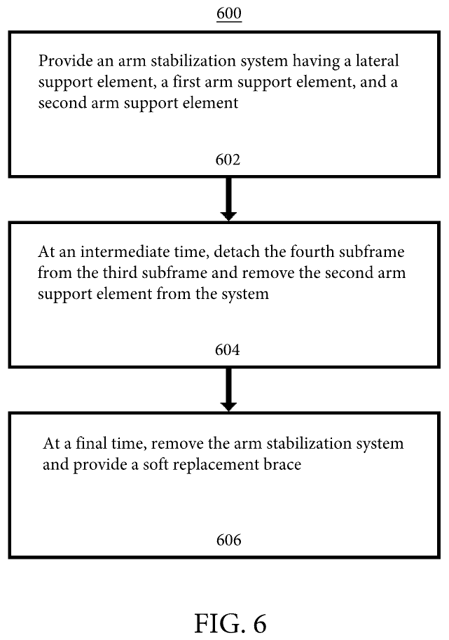

FIG. 6 shows a method 600 of stabilizing an arm, according to an embodiment.

The method 600 may begin at step 602 in which, at an initial time, an arm stabilization system 100 of FIG. 1B is provided. The arm stabilization system 100 has a lateral support element 102, a first arm support element 104, and a second arm support element 106.

The method 600 may proceed to step 604 in which, at an intermediate time, the fourth subframe 308 is detached from the third subframe 306 and the second arm support element 106 is removed from the arm stabilization system 100.

The method may optionally proceed to step 606 in which, at a final time, the arm stabilization system 100 is removed and replaced with a soft brace. The intermediate and final times may be selected based on wearer needs.

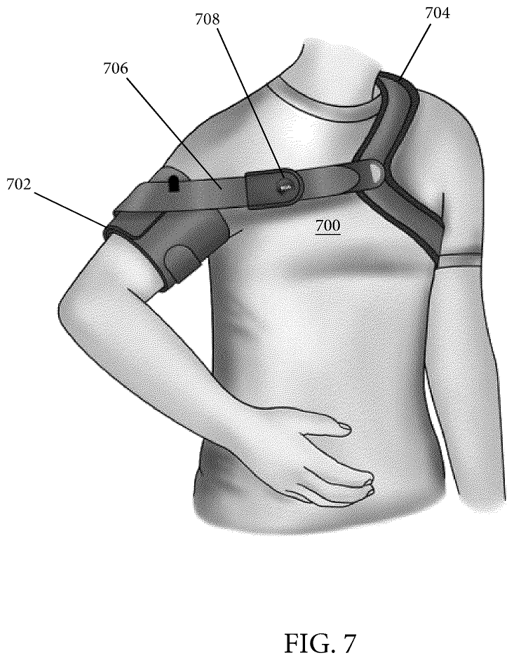

FIG. 7 shows an arm stabilization system 700, according to a third embodiment. The system 700 may be comprised of an upper arm support element 702, an opposing support element 704, a first strap 706, and, optionally, a second strap (not shown) and a third strap (not shown).

The upper arm support element 702 is configured to support the upper arm, and to receive first ends of the first 706 and second straps. In a preferred embodiment, the upper arm support element 702 is comprised of soft padding that encircles the upper arm.

The opposing support element 704 is configured to contact the torso at an opposite end to the upper arm support element 702, and to receive second ends of the first 706 and second straps.

The first 706, second, and third straps may be comprised of a weakly elastic material, a strongly elastic material, a non-elastic material, or a combination thereof. The path of the first strap 706 starts on the upper arm support element 702, spirals clockwise around the upper arm support element 702, and terminates on the opposing support element 702. The path of the second strap (not shown) starts on the upper arm starts on the upper arm support element 702, spirals counterclockwise around the upper arm support element 702, and terminates on the opposing support element 702. Preferably, the second strap (not shown) spirals in an opposite direction to the first strap 706 and the paths of the first 706 and second straps traverse the sagittal plane.

Tensioning mechanisms 708 may be positioned on the first 706, second, and third straps, or on the arm stabilization system 700 adjacent to the first 706, second, and third straps.

In an alternative embodiment (not shown), the opposing support element 704 is configured to encircle the waist, and to receive second ends of the first 706, second, and third straps. The path of the third strap starts on the upper arm support element 702 and terminates on the opposing support element 704.

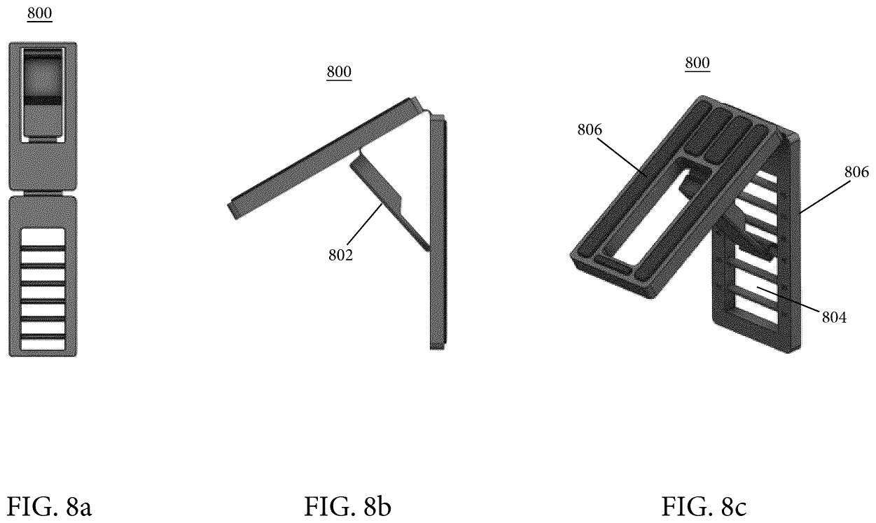

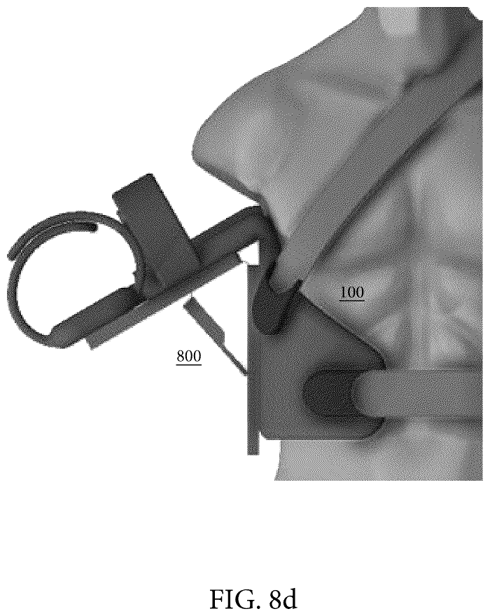

FIGS. 8a-8d show an adjustable abduction wedge 800, according to an embodiment. Namely, FIG. 8a shows the adjustable abduction wedge 800 in an open configuration, FIG. 8b shows the adjustable abduction wedge 800 from a side view, FIG. 8c shows the adjustable abduction wedge 800 from a perspective view, and FIG. 8d shows the adjustable abduction wedge 800 used in conjunction with the arm stabilization system 100.

Placing the wedge 802 into a desired slot 804 can be used to adjust the angle in between the wedge halves. One part of a two-part fastening mechanism 508 may be attached, embedded, or otherwise integrated into inner surfaces of the wedge halves. For example, hooks may cover the inner surfaces of the wedge halves while loops may cover the outer surfaces of the arm stabilization system 100, enabling the adjustable abduction wedge 800 to be attached to the arm stabilization system 100, as shown in FIG. 8d.

While specific arm stabilization systems and methods been contemplated, it would be readily appreciated by those knowledgeable in the field that the novel support and subframe elements, individually, or in any combination, may be applied to stabilize other parts of the body.

We claim:

1. An arm stabilization system, the system comprising:
a lateral support element configured to contact a side of a torso, wherein
a first subframe is embedded in the lateral support element;
a second subframe, slidably attached to the first subframe, is embedded in the lateral support element;
a first arm support element configured to contact an upper arm, wherein
a third subframe, rotatably attached to the second subframe via a multiaxial joint, is embedded in the first arm support element;
a fourth subframe, slidably attached to the third subframe, is embedded in the first arm support element;
wherein
a first distance between the first and second subframes is adjustable;
a second distance between the third and fourth subframes is adjustable;
the third subframe is rotatable around first and second axes, wherein
the first axis is defined by a line passing through the multiaxial joint and a distal end of the fourth subframe; and
the second axis, perpendicular to the first axis, is defined by a horizontal line passing through the multiaxial joint.

2. The system of claim 1, further comprising:
a second arm support element configured to contact a lower arm, wherein
a fifth subframe, slidably attached to the fourth subframe, is embedded in the second arm support element; and
wherein
a third distance between the fourth and fifth subframes is adjustable.

3. The system of claim 1, further comprising:
at least one of a first locking mechanism and a second locking mechanism, wherein
the third subframe is not rotatable around the first axis when the first locking mechanism is engaged; and
the third subframe is not rotatable around the second axis when the second locking mechanism is engaged.

4. The system of claim 1, wherein
the lateral support element is securable around a waist.

5. The system of claim 4, wherein
the lateral support element is secured to the waist via a shape-maintaining moldable strap.

6. The system of claim 2, wherein
the first and second support elements are securable around the upper and lower arm.

7. The system of claim 6, wherein
the first arm support is secured to the upper arm via a shape-maintaining moldable strap.

8. The system of claim 7, wherein
the system is reconfigurable such that the lateral support element is configured to contact an opposite side of the torso and the first arm support element is configured to contact an opposite upper arm.

9. An arm stabilization system, the system comprising:
a lateral support element configured to contact a side of a torso, wherein
a first subframe is embedded in the lateral support element;
a second subframe, slidably attached to the first subframe, is embedded in the lateral support element;
a first arm support element configured to contact an upper arm, wherein
a third subframe, rotatably attached to the second subframe via a multiaxial joint, is embedded in the first arm support element;
a fourth subframe, slidably attached to the third subframe, is embedded in the first arm support element;
a fifth subframe, slidably attached to the fourth subframe, is embedded in the second arm support element;
wherein
a first distance between the first and second subframes is adjustable;
a second distance between the third and fourth subframes is adjustable;
a third distance between the fourth and fifth subframes is adjustable;
the third subframe is rotatable around first and second axes, wherein
the first axis is defined by a line passing through the multiaxial joint and a distal end of the fourth subframe; and
the second axis, perpendicular to the first axis, is defined by a horizontal line passing through the multiaxial joint.

10. The system of claim 9, further comprising:
first and second locking mechanisms, wherein
the third subframe is not rotatable around the first axis when the first locking mechanism is engaged; and
the third subframe is not rotatable around the second axis when the second locking mechanism is engaged.

11. The system of claim 10, wherein
the lateral support element is secured to a waist via a first shape-maintaining moldable strap; and
the first and second arm support elements are secured to the upper arm via a second shape-maintaining moldable strap.

12. The system of claim 10, wherein
the system is reconfigurable to such that the lateral support element is configured to contact an opposite side of the torso and the first arm support element is configured to contact an opposite upper arm.

13. A method of stabilizing an arm, the method comprising steps of:
at an initial time, providing an arm stabilization system, the system comprising:
a lateral support element configured to contact a side of a torso, wherein
a first subframe is embedded in the lateral support element;
a second subframe, slidably attached to the first subframe, is embedded in the lateral support element;
a first arm support element configured to contact an upper arm, wherein
a third subframe, rotatably attached to the second subframe via a multiaxial joint, is embedded in the first arm support element;
a fourth subframe, slidably attached to the third subframe, is embedded in the first arm support element;
a second arm support element configured to contact a lower arm, wherein
a fifth subframe, slidably attached to the fourth subframe, is embedded in the second arm support element;
wherein
a first distance between the first and second subframes is adjustable;
a second distance between the third and fourth subframes is adjustable;
a third distance between the fourth and fifth subframes is adjustable;
the third subframe is rotatable around first and second axes, wherein
the first axis is defined by a line passing through the multiaxial joint and a distal end of the fourth subframe; and
the second axis, perpendicular to the first axis, is defined by a horizontal line passing through the multiaxial joint; and
at an intermediate time, detaching the fourth support element from the third support element and removing the second arm support element.

14. The method of claim 13, further comprising a step of:
at the intermediate time, slidably attaching a sixth support element to the third support element, wherein
a fourth distance between the third and sixth subframes is adjustable.

15. The method of claim 13, wherein the arm stabilization system further comprises:
first and second locking mechanisms, wherein
the third subframe is not rotatable around the first axis when the first locking mechanism is engaged; and
the third subframe is not rotatable around the second axis when the second locking mechanism is engaged.

16. The method of claim 13, wherein
the lateral support element is securable around a waist.

17. The method of claim 13, wherein
the lateral support element is secured to the waist via a shape-maintaining moldable strap.

18. The system of claim 13, wherein
the first and second support elements are securable around the upper arm.

19. The system of claim 13, wherein
the first arm support is secured to the upper arm via a shape-maintaining moldable strap.

20. The system of claim 13, wherein
the arm stabilization system is reconfigurable such that the lateral support element is configured to contact an opposite side of the torso and the first arm support element is configured to contact an opposite upper arm.
